(19) United States
(12) Patent Application Publication
(10) Pub. No.: US 2023/0133564 A1
(43) Pub. Date: May 4, 2023

(54) CD3-FUSION PROTEIN AND USES THEREOF

(71) Applicant: MEDIGENE IMMUNOTHERAPIES GMBH, Planegg-Martinsried (DE)

(72) Inventors: Nadja SAILER, Muenchen (DE); Christian ELLINGER, Muenchen (DE)

(21) Appl. No.: 17/916,224

(22) PCT Filed: Mar. 29, 2021
(86) PCT No.: PCT/EP2021/058124

§ 371 Date: Sep. 30, 2022

(30) Foreign Application Priority Data
Apr. 1, 2020 (EP) 20167540.2

(51) Int. Cl. C07K 14/725; C07K 14/705; C07K 16/28; A61K 35/17

(52) U.S. Cl. CPC C07K 14/7051; C07K 14/70517; C07K 14/70521; C07K 16/2809; C07K 16/2818; A61K 35/17; C07K 2319/00

(57) ABSTRACT

The present invention provides tools and methods for the TCR independent activation of T-cells, in particular TCR negative T-cells. In particular, the invention relates to CD3-fusion protein comprising a CDS heterodimer comprising a transmembrane domain, and a CD3 domain. The invention further relates to a nucleic acid molecule encoding such a CD3-fusion protein, a T cell encoding such a CD3-fusion protein as well as said T cell for medical use. Further, the use of the T cell for testing and characterization of exogenous effector molecules is described.

### FIELD OF THE INVENTION

The present invention provides tools and methods for the TCR independent activation of T-cells, in particular TCR negative T-cells. In particular, the invention relates to CD3-fusion proteins comprising a CD3ε ectodomain, a CD3δ or CD3γ ectodomain, a transmembrane domain, and a CD3ζ domain. The invention further relates to a nucleic acid molecule encoding such CD3-fusion proteins, a T cell encoding such CD3-fusion proteins as well as said T cell for medical use. Further, the use of the T cell for testing and characterization of exogenous effector molecules is described.

### BACKGROUND OF THE INVENTION

T lymphocytes are part of the adaptive immune response and originate from hematopoietic stem cells located in the bone marrow. T lymphocytes express a unique antigen binding receptor on their membrane, the T cell receptor (TCR), which recognizes antigens in association with major histocompatibility complex (MHC) molecules.

With their central role in the immune system, T cells typically provide protection from pathogens or malignant cells. Each T cell expresses a single form of a T cell receptor (TCR)—a structure which is used by the T cell to recognize infected or altered cells.

The concept of immunotherapy is based on the specificity of the adaptive immune response for the recognition and elimination of pathogens as well as tumor cells. The aim of a successful immunotherapy is the manipulation or reprogramming of the patient's immune response in order to specifically target tumor cells for destruction by the immune system.

Therapeutic approaches used to reprogram the immune system in the treatment of cancer include active immunotherapy comprising the use of vaccination strategies, including DC vaccines, as well as passive immunotherapy comprising the application of tumor-specific antibodies or genetically engineered lymphocytes or the adoptive transfer of T cells specifically recognizing tumor antigens.

The principle of adoptive T cell transfer is based on the ex vivo expansion of autologous or allogeneic tumor-specific T lymphocytes and the subsequent re-infusion into patients. Cancer regression in patients suffering from metastatic melanoma has been observed after the transfer of ex vivo expanded autologous tumor-infiltrating lymphocytes (TILs). The drawback of this therapeutic approach is the requirement for pre-existing tumor-reactive cells that need to be isolated from every individual patient as well as the difficult detection of TILs for cancers other than melanoma. Therefore, other methods were developed that focus on the genetic modification of T cells isolated from patients. These genetically engineered T cells can for example be created by transduction of autologous T cells with the α and β chains of tumor-specific TCRs, i.e. with recombinant TCRs.

An approach, as developed by Wilde et al. in 2009 and described in WO 2007/017201, allows the isolation of allo-restricted peptide-specific T cells using autologous DCs co-transfected with RNA species encoding both the TAA and a selected allogeneic MHC molecule. By co-culturing autologous T cells with DCs presenting self-peptide/allo-MHC complexes, high-avidity T cells that recognize self-antigen can be obtained (Wilde et al., 2009, Dendritic cells pulsed with RNA encoding allogeneic MHC and antigen induce T cells with superior antitumor activity and higher TCR functional avidity. Blood, 114(10), 2131-9). Because T cell cultivation and expansion of T cell clones is laborious and requires repeated rounds of re-stimulation, it is an advantage to isolate cDNAs of the TCRs at an early time point in order to allow their characterization by introducing them into recipient PBL. This allows the characterization of the TCRs regarding antigen specificity, avidity and functionality before they are used for therapeutic application in patients. However, this method is laborious and time consuming. Additionally, the endogenous TCRs of the lymphocytes, having unknown specificity, can form heterodimers with the introduced transgenic/recombinant TCRs, leading to cross-reactivity of again unknown specificity.

In addition, in light of the substantial progress that has been made in adoptive immunotherapy for cancer, there is a need for the provision of a decisive and quick method to test and characterize transgenic TCRs for their potential for use in adoptive T cell therapy.

It is a prerequisite of T cell development that the T cell can proliferate, so that it can be further propagated. However, the proliferation of T cells is inter alia dependent on the expression of an intact TCR. Hence, in the recipient cell, which is devoid of a functional TCR, there is a need for an alternative proliferation signal that allows the recipient T cell to divide in the absence of a functional TCR. In particular, it is necessary that the proliferation of the T cell can be triggered in vitro and/or in vivo.

Further, it is desirable when used as a therapeutic agent, that the recipient cell can proliferate in vitro or in vivo independently of the receptor mediating the therapeutic effect. This allows the propagation of the recipient cell before its therapeutic fate is determined. In addition, this approach allows the uniform induction of a proliferation stimulus in vitro and/or in vivo after the therapeutically active receptor is introduced. Thereby, different therapeutic cell lines, each carrying a different therapeutic receptor, such as a TCR, can still be stimulated in a uniform and standardized manner by the proliferation stimulus which is independent of the molecule mediating the therapeutic effect.

Thus, straight forward strategies are needed for the culturing of T cells independent of a stimulus of their endogenous TCR.

### OBJECTIVES AND SUMMARY OF THE INVENTION

The present invention provides strategies for the propagation of T cells independent of the TCR. In particular the invention provides a CD3-fusion protein comprising:
a CD3ε ectodomain,
a CD3δ ectodomain or CD3γ ectodomain,
a transmembrane domain, and
a CD3ζ domain.

This CD3-fusion protein, when expressed in T cells, allows the TCR independent activation of the T cell upon a CD3 and CD28 activation stimulus. In particular, the T cells can be easily activated by a composition comprising anti-CD3 and anti-CD28 antibodies. Thus, the TCR independent activation of the T cells can be carried out using commercially available products, such as microsphere beads on which anti-CD3 and anti-CD28 antibodies are immobilized. Thus, the time-consuming activation using feeder cells, such as LCL cells becomes unnecessary. Thereby straight forward economic methods for the TCR independent activation of TCRs are provided. Since the CD3-fusion protein does not recognize any specific target in vitro, background activation in cellular assays is reduced.

Typically, the mentioned transmembrane domain is a CD28 transmembrane domain. The CD3 ectodomains may be linked to the transmembrane domain by a hinge domain. The hinge domain linking the CD3 ectodomains to the transmembrane domain may be selected from the group consisting of IgG hinge domain, CD28 hinge domain or CD8 hinge domain.

The hinge domain linking the CD3 ectodomains to the transmembrane domain may be selected from the group consisting of IgG hinge domain, CD28 hinge domain or CD8 hinge domain. Preferably, the hinge domain is a CD8 hinge domain.

The fusion protein further may further comprise a signal peptide domain allowing the co-translational localization to the ER membrane. The signal domain is preferably a CD8 signal domain.

Typically, the CD3δ ectodomain and the CD3ε ectodomain or the CD3ε ectodomain and the CD3γ ectodomain are connected via a linker, preferably a non-immunogenic linker. Typically, the linker comprises at least 5 amino acids. The amino acids may be selected from the group of glycine and serine residues.

In a preferred embodiment, the CD3-fusion protein comprises
a CD8 signal peptide domain,
a CD3δ ectodomain and a CD3ε ectodomain,
a CD8 hinge domain,
a CD28 transmembrane domain,
a CD3ζ domain.

The CD8 signal peptide may comprise an amino acid sequence which is SEQ ID NO: 1 or at least 80% identical to SEQ ID NO: 1.

The CD3δ ectodomain may comprise an amino acid sequence which is SEQ ID NO: 2 or at least 80% identical to SEQ ID NO: 2.

The CD3γ ectodomain may comprise an amino acid sequence which is SEQ ID NO: 3 or which is at least 80% identical to SEQ ID NO: 3.

The CD3ε ectodomain may comprise an amino acid sequence which is SEQ ID NO: 4 or which is at least 80% identical to SEQ ID NO: 4.

The linker connecting the CD3δ ectodomain and the CD3c ectodomain or the CD3δ domain and the CD3γ ectodomain may comprise an amino acid sequence which is SEQ ID NO: 5 or which is at least 80% identical to SEQ ID NO: 5.

The CD8 hinge domain may comprise an amino acid sequence which is SEQ ID NO: 6 or which is at least 80% identical to SEQ ID NO: 6.

The transmembrane domain may comprise an amino acid sequence which is SEQ ID NO: 7 or which is at least 80% identical to SEQ ID NO: 7.

The CD3ζ domain may comprise an amino acid sequence which is SEQ ID NO: 8 or which is at least 80% identical to SEQ ID NO: 8.

In a specific embodiment the fusion protein may comprise an amino acid sequence which is SEQ ID NO: 11 or at least 80% identical to SEQ ID NO: 11.

Typically, the order of the domains in the direction of N- to C-terminus of the CD3-fusion protein is a CD3δ ectodomain or CD3γ ectodomain, linker, CD3ε ectodomain, hinge domain, CD28 transmembrane domain and CD3ζ domain.

The CD3-fusion protein may further comprise at least one co-stimulatory molecule selected from the group consisting of CD28, Ox40, ICOS and CD28.

A further aspect of the invention refers to a nucleic acid molecule containing a sequence which encodes for the CD3-fusion protein as described herein.

The nucleic acid molecule may further comprise a sequence encoding a fluorescence protein. Typically, between the sequence encoding the CD3-funsion protein and the sequence encoding the fluorescence protein there is a ribosomal skipping sequence.

Another aspect of the invention refers to the use of the CD3-fusion protein as described herein or of the nucleic acid molecule as described herein for activation of TCR-negative T cells by a CD3-stimulus and a CD28-stimulus. Preferably, the CD3-stimulus is an activating anti-CD3 antibody or a binding fragment thereof.

The CD28 stimulus may be in the form of co-culture with feeder cells, e.g. Lymphoblastoid cell lines (LCL) cells or by activating anti-CD28 antibody. Preferably, the CD28-stimulus is an activating anti-CD28 antibody or a binding fragment thereof.

Preferably, for the TCR independent activation, a composition comprising an anti-CD3 and an anti-CD28 antibody is used. The anti-CD3 and anti-CD28 antibodies may be immobilized to suitable surface, e.g. on the surface of microsphere beads, on Streptamers® or on a tissue culture vessel surface. Preferably, the composition comprising anti-CD3 and anti-CD28 antibodies or binding fragments thereof is immobilized on microsphere beads.

Thus, the present method is advantageous, since no cytokines and no feeder cells are necessary for stimulation resulting in a cheaper and more efficient stimulation.

Another aspect of the invention refers to a method for TCR independent activation of T cells comprising the steps:
Expressing the CD3-fusion protein as described herein,
Stimulating the T cells with a CD3- and a CD28-stimulus.

The method may also comprise the step of deletion of the endogenous TCR. Thus, the method may comprise the following steps:
Expressing the CD3-fusion protein as described herein,
Deleting the endogenous TCR
Stimulating the T cells with a CD3- and a CD28-stimulus.

Another aspect of the invention refers to a T cell comprising the CD3-fusion protein as described herein.

The skilled person understands that the T cell is CD28-positive, i.e. the T cell expresses CD28 on its cell surfaces.

The goal of the TCR-independent T cell activation is to keep the T cells devoid of an expressed TCR viable during cell culture, i.e. to expand the T cells in cell culture. Upon the expression of a transgenic/recombinant TCR or chimeric antigen receptor, the T cells elicit cellular effector function (i.e. IFN-γ release) and killing capacity. Thus, typically the TCR complex is knocked out or its expression is suppressed at one stage of the method of the invention. Therefore, in some embodiments, the T cells comprising the CD3-fusion protein as described herein do not express a functional TCR, or in other word are TCR receptor negative.

Such TCR-negative T cells comprising the CD3-fusion protein as described herein are ready to use for the introduction of exogenous receptors which are able to activate the immune effector functions of the recipient cell.

The invention also refers to the T cell for use as a medicament. In particular, the invention refers to a T cell for the treatment of cancer.

### FIGURE LEGENDS

FIG. 1: Domain structure of a CD3-fusion protein. The extracellular single chain fragment (scFv) of a CD3δ ectodomain and a CD3ε ectodomain separated by a flexible (Gly₄Ser)₃ linker is coupled to a CD28 transmembrane domain and a CD3ζ signaling domain via a CD8 hinge domain. The scFv retains the binding epitope of the α-CD3 antibody OKT-3 upon native folding. The coding sequence for the CD3-fusion protein is preceded by a CD8 signal peptide to ensure co-translational localization to the ER (endoplasmic reticulum) membrane. eGFP coupled via P2A element can be used to monitor successful transduction into cells. Multiple cloning sites (MCS) allow easy cloning into different backbone vectors.

FIG. 2: Strategy for utilizing CD3-fusion proteins. T cells transduced with a CD3-fusion protein can be activated by α-CD3 and α-CD28 antibodies to deliver a stimulation signal 1 and 2 in the absence of the endogenous TCR after knockout. Antibodies binding the CD3-fusion protein can either be used in soluble form in the presence of feeder cells or in bead-bound (microspheres) form in combination with an α-CD28 antibody.

FIG. 3: CD3-fusion protein expression in TCR-deficient Jurkat-76 cells. Transduced Jurkat-76 cells were analyzed for the expression of CD3-fusion protein and eGFP expression 7 days after transduction by staining with α-CD3 antibody and subsequent flow cytometric analysis. Generated single cell clones (single cell cloning) were subsequently also evaluated for the expression of the CD3-fusion protein and eGFP by α-CD3 antibody staining followed by flow cytometric analysis 2 weeks after FACS. Results of 2 representative clones are shown. Transduced cells are depicted in grey. Untransduced Jurkat-76 cells served as negative controls (black line). Within histograms, percentage of CD3 positive cells and respectively eGFP positive cells is shown.

FIG. 4: X-Fold expansion of TCR-deficient T cell clones transduced with CD3-fusion protein. a) X-Fold expansion of four different T cell clones (CD8+_001, CD8+_002, CD8+_003 and CD8+_004), over the course of 56 days with repeated stimulation every 14 days. Cell count was determined after each expansion round. Clone CD4+_50 served as a control (Control), carrying its endogenous TCR, to compare proliferative capacity. b) X-Fold expansion of two selected T cell clones over a single stimulation cycle using different activation conditions. Cells were either stimulated with complete stimulation mix (black filled rectangulars or black filled circles) comprising LCL feeder cells, IL-2 and OKT-3 antibody or with stimulation mix lacking OKT-3 (grey filled rectangulars or grey filled circles) or OKT-3 and LCL (unfilled rectangulars or unfilled circles), respectively. The number of viable cells was determined on day 5 and 10.

FIGS. 5a, 5b and 5c: Effector function of CD3-fusion protein-transduced T cell clones after transduction with either tyrosinase-specific TCRs T58 or D115. FIG. 5a) Staining of isolated T cell clones (CD3-fusion protein 001=CD8+_001 and CD3-fusion protein 002=CD8+_002) and PBL transduced either with T58 or D115 , respectively, with α-CD3 and α-TCR-Vβ antibodies specific for the respective transgenic TCR β chain and tetramer comprising the YMDGTMSQV (YMD) peptide. Numbers shown represent percentage of cells being CD3-positive and at the same time binding a specific TRBV antibody or the respective tetramer. After enrichment by FACS, cells were subsequently analyzed by flow cytometric analysis. Untransduced CD8+_001 and CD8+_002 T cell clones and untreated PBL were used as respective negative controls (depicted in gray in histograms). FIG. 5b) IFN-γ ELISA of supernatants derived from the same co-cultures used in the killing assay 20 hours after incubation of transduced T cells (CD3-fusion protein 001=CD8+_001, CD3-fusion protein 002=CD8+_002 or PBL) with target cells at an E:T ratio of 2:1. Positive control comprised effector cells activated with 750 ng/mL PMA and 5 ng/mL ionomycin (PMA/Iono). IFN-γ release of transduced PBL was determined in two independent experiments. Untransduced cells (w/o) served as negative control. Target cells comprised tyrosinase-positive (Mel624.38) and tyrosinase-negative tumor cells (A375). FIG. 5c) Functional avidity of TCR-transduced T cell clones (CD3-fusion protein 001=CD8+_001, CD3-fusion protein 002=CD8+_002) and PBL plotted as relative IFN-γ release in response to decreasing peptide concentrations. K562_A2_CD86 cells were loaded with decreasing amounts of peptide (10⁻⁴ to 10⁻¹² M) and co-cultured with T58 or D115-expressing T cells, respectively, at a fixed E:T ratio of 2:1. IFN-γ release was determined 20 hours later by IFN-γ ELISA and relative IFN-γ release was calculated by setting maximal IFN-γ release to the reference value of 100%. Lower values were calculated according to this reference. Values were derived from biological duplicates. Dashed lines indicate calculated EC50 values. K652_A2_CD86 cells loaded with 10⁻⁴ M irrelevant SLLMWITQC peptide (SEQ ID NO: 25) served as negative controls.

FIG. 6: Killing capacity of isolated CD3-fusion protein expressing T cell clones after transduction of either transgenic tyrosinase-specific TCR T58 (001_T58) or D115 (001_D115). Killing assay was conducted using the IncuCyte® ZOOM System to monitor killing (cytolysis) of tyrosinase-positive (Mel624.38) and -negative (A375) tumor cells labeled with IncuCyte® NucLight Red dye over 72 hours with E:T ratio of 2:1. Respective tumor cells alone (unfilled circles) served as control for proliferation in the absence of effector cells. Untransduced effector cells (unfilled squares, 001) served as control to estimate background killing in the absence of transgenic TCRs. Cell count/well was determined by using the IncuCyte® ZOOM Software 2016B to analyze quadruplicates of each approach.

FIG. 7: X-Fold Expansion of T cell clones (001 and 002) transduced with CD3-fusion protein, either stimulated using stimulation via feeder cells (LCL cells) and α-CD3 antibody or with α-CD3/CD28 antibody coated microsphere beads or stimulation via CD3/CD28 streptamer. X Fold Expansion of two different T cell clones (001 and 002) is shown.

### DETAILED DESCRIPTION OF THE INVENTION

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures, but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun, e.g. “a”, “an” or “the”, this includes a plural of that noun unless something else is specifically stated.

Technical terms are used by their common sense. If a specific meaning is conveyed to certain terms, definitions of terms will be given in the following in the context of which the terms are used.

The present invention provides strategies for the propagation of T cells independent of the TCR. In particular, the invention provides a CD3-fusion protein comprising:
a CD3ε ectodomain,
a CD3δ ectodomain or CD3γ ectodomain, and
a transmembrane domain, and
a CD3ζ domain.

Typically, the transmembrane domain is a CD28 transmembrane domain. The CD3ε ectodomain and CD3δ ectodomain or CD3γ ectodomain may be linked to the transmembrane domain by a hinge domain. The hinge domain linking the CD3ε ectodomain and CD3δ ectodomain or CD3γ ectodomain to the transmembrane domain may be selected from the group consisting of IgG hinge domain, CD28 hinge domain or CD8 hinge domain.

CD3-fusion protein according to claims 1 to 3 wherein the hinge domain linking the CD3ε ectodomain and CD3δ ectodomain or CD3γ ectodomain to the transmembrane domain is selected from the group consisting of IgG hinge domain, CD28 hinge domain or CD8 hinge domain. Preferably, the hinge domain is a CD8 hinge domain.

The fusion protein further may further comprise a signal peptide domain allowing the co-translational localization to the ER membrane. The signal domain is preferably a CD8 signal domain.

Typically, the CD3δ ectodomain and the CD3ε ectodomain or the CD3ε ectodomain and the CD3γ ectodomain are connected by a preferably non-immunogenic linker. Typically, the linker contains at least 5 amino acids. The amino acids may be selected from the group of glycine and serine residues.

In a preferred embodiment, the CD3-fusion protein comprises
a CD8 signal peptide domain,
a CD3δ ectodomain and a CD3ε ectodomain,
a CD8 hinge domain,
a CD28 transmembrane domain,
a CD3ζ domain.

The CD8 signal peptide may comprises an amino acid sequence which is SEQ ID NO: 1 or at least 80% identical to SEQ ID NO: 1.

The CD3δ ectodomain may comprise an amino acid sequence which is SEQ ID NO: 2 or at least 80% identical to SEQ ID NO: 2.

The CD3γ ectodomain may comprise an amino acid sequence which is SEQ ID NO: 3 or which is at least 80% identical to SEQ ID NO: 3.

The CD3ε ectodomain may comprise an amino acid sequence which is SEQ ID NO: 4 or which is at least 80% identical to SEQ ID NO: 4.

The linker connecting the CD3δ ectodomain and the CD3ε ectodomain or the CD3δ ectodomain and the CD3γ ectodomain may comprise an amino acid sequence which is SEQ ID NO: 5 or which is at least 80% identical to SEQ ID NO: 5.

The CD8 hinge domain may comprise an amino acid sequence which is SEQ ID NO: 6 or which is at least 80% identical to SEQ ID NO: 6.

The transmembrane domain may comprise an amino acid sequence which is SEQ ID NO: 7 or which is at least 80% identical to SEQ ID NO: 7.

The CD3 domain may comprise an amino acid sequence which is SEQ ID NO: 8 or which is at least 80% identical to SEQ ID NO: 8.

In a specific embodiment the fusion protein may comprise an amino acid sequence which is SEQ ID NO: 11 or at least 80% identical to SEQ ID NO: 11.

“At least 80% identical”, in particular “having an amino acid sequence which is at least 80% identical” as used herein includes that the amino acid sequence is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set out.

The determination of percent identity between multiple sequences is preferably accomplished using the AlignX application of the Vector NTI Advance™ 10 program (Invitrogen Corporation, Carlsbad Calif., USA). This program uses a modified Clustal W algorithm (Thompson et al., 1994. Nucl Acids Res. 22: pp. 4673-4680; Invitrogen Corporation; Vector NTI Advance™ 10 DNA and protein sequence analysis software. User's Manual, 2004, pp.389-662). The determination of percent identity is performed with the standard parameters of the AlignX application.

Typically, the order of the domains in the direction of N-terminus to C-terminus of the fusion protein is CD3δ ectodomain or CD3γ ectodomain, CD3ε domain, hinge domain, CD28 transmembrane domain, CD3ζ domain.

The CD3-fusion protein may further comprise at least one co-stimulatory molecule selected from the group consisting of CD28, Ox40, ICOS and CD28.

The term “propagation of T cells independent of the TCR” refers to a T cell that proliferates without the need of the stimulation of a TCR. It is understood that the “human T cell which proliferates independent of a proliferation stimulus from a TCR” may express a TCR and therefore it may be possible that the T cell proliferates in addition based on the proliferation stimulus from a TCR.

A TCR is composed of two different and separate protein chains, namely the TCR alpha (a) and the TCR beta (b) chain. The TCR α chain comprises variable (V), joining (J) and constant (C) regions. The TCR b chain comprises variable (V), diversity (D), joining (J) and constant (C) regions. The rearranged V(D)J regions of both the TCR α and the TCR β chain contain hypervariable regions (CDR, complementarity determining regions), among which the CDR3 region determines the specific epitope recognition. At the C-terminal region both TCR α chain and TCR β chain contain a hydrophobic transmembrane domain and end in a short cytoplasmic tail.

Typically, the TCR is a heterodimer of one α chain and one β chain. This heterodimer can bind to MHC molecules presenting a peptide.

The term “variable TCR α region” or “TCR α variable chain” or “variable domain” in the context of the invention refers to the variable region of a TCR α chain. The term “variable TCR β region” or “TCR β variable chain” in the context of the invention refers to the variable region of a TCR β chain.

The TCR loci and genes are named using the International Immunogenetics (IMGT) TCR nomenclature (IMGT Database, www. IMGT.org; Giudicelli, V., et al., IMGT/LIGM-DB, the IMGT® comprehensive database of immunoglobulin and T cell receptor nucleotide sequences, Nucl. Acids Res., 34, D781-D784 (2006). PMID: 16381979; T cell Receptor Factsbook, LeFranc and LeFranc, Academic Press ISBN 0-12-441352-8).

A further aspect of the invention refers to a nucleic acid molecule containing a sequence which encodes for the CD3-fusion protein as described herein.

The nucleic acid molecule may further comprise a sequence encoding a fluorescence protein. Typically, between the sequence encoding the CD3-funsion protein and the sequence encoding the fluorescence protein there is a ribosomal skipping sequence.

The following table indicates the nucleotide sequences encoding the respective peptide sequences:

| Peptide | Nucleotide | |
|---|---|---|
| sequence | sequence | |
| SEQ ID NO | SEQ ID NO | description |
| 1 | 12 | CD8 signal peptide domain |
| 2 | 13 | CD3δ ectodomain |
| 3 | 14 | CD3γ ectodomain |
| 4 | 15 | CD3ε ectodomain |
| 5 | 16 | (G4S) 3 linker aa |
| 6 | 17 | CD8 hinge domain |
| 7 | 18 | CD28 transmembrane domain |
| 8 | 19 | CD3ζ domain |
| 9 | 20 | Spacer P2A aa |
| 10 | 21 | eGFP aa |
| 11 | 22 | CD3-Chimera complete sequence |
| | | aa CD3δ/ε |
| 23 | 24 | CD3-Chimera complete sequence |
| | | aa CD3γ/ε |

“Nucleic acid molecule” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids can be constructed based on chemical synthesis and/or enzymatic ligation reactions using procedures known in the art or commercially available (e.g. from Genscript, Thermo Fisher and similar companies). See, for example Sambrook et al., a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). The nucleic acid can comprise any nucleotide sequence which encodes any of the recombinant TCRs, polypeptides, or proteins, or functional portions or functional variants thereof.

The present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence encoding the CD3-fusion protein described herein.

The disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand and are particularly suitable for detecting expression of any of the TCRs described herein. It is generally appreciated that conditions can be rendered more stringent by the addition of increasing amounts of formamide.

As already described elsewhere herein, the nucleic acid encoding the TCR may be modified. Useful modifications in the overall nucleic acid sequence may be codon optimization. Alterations may be made which lead to conservative substitutions within the expressed amino acid sequence. These variations can be made in complementarity determining and non-complementarity determining regions of the amino acid sequence of the TCR chain that do not affect function. Usually, additions and deletions should not be performed in the CDR3 region.

Another embodiment refers to a vector comprising the nucleic acid encoding the CD3-fusion protein as described herein.

The vector is preferably a plasmid, shuttle vector, phagemide, cosmid, expression vector, retroviral vector, adenoviral vector or particle and/or vector to be used in gene therapy.

A “vector” is any molecule or composition that has the ability to carry a nucleic acid sequence into a suitable host cell where synthesis of the encoded polypeptide can take place. Typically, and preferably, a vector is a nucleic acid that has been engineered, using recombinant DNA techniques that are known in the art, to incorporate a desired nucleic acid sequence (e.g. a nucleic acid of the invention). The vector may comprise DNA or RNA and/or comprise liposomes. The vector may be a plasmid, shuttle vector, phagemide, cosmid, expression vector, retroviral vector, lentiviral vector, adenoviral vector or particle and/or vector to be used in gene therapy. A vector may include nucleic acid sequences that permit it to replicate in a host cell, such as an origin of replication. A vector may also include one or more selectable marker genes and other genetic elements known to those of ordinary skill in the art. A vector preferably is an expression vector that includes a nucleic acid according to the present invention operably linked to sequences allowing for the expression of said nucleic acid.

Preferably, the vector is an expression vector. More preferably, the vector is a retroviral, more specifically a gamma-retroviral or lentiviral vector.

Another aspect of the invention refers to the use of the fusion protein as described herein or of the nucleic acid molecule as described herein for activation of TCR-negative T cells by a CD3 stimulus and a CD28 stimulus. Preferably, the CD3 stimulus is an activating anti-CD3 antibody or a binding fragment thereof.

The CD28 stimulus may be lymphoblastoid cell lines (LCL) cells (which typically activate CD28 via CD86 and or CD80) or an activating anti-CD28 antibody. Preferably, the CD28 stimulus is an activating anti-CD28 antibody.

Preferably, for the TCR independent activation a composition comprising an anti-CD3 and an anti-CD28 antibody or a binding fragment thereof, e.g. a Fab-fragment is used. The anti-CD3 and anti-CD28 antibodies or binding fragment thereof may be immobilized, e.g. on beads, on Streptamers® or on a tissue culture vessel surface. Preferably, the composition comprising anti-CD3 and anti-CD28 is immobilized on beads.

Binding fragments may thus include portions of an intact full-length antibody, such as an antigen binding or variable region of the complete antibody. Examples of antibody fragments include Fab, F(ab′)2, Id and Fv fragments; diabodies; linear antibodies; single-chain antibody molecules (e.g., scFv); multispecific antibody fragments such as bispecific, trispecific, and multispecific antibodies (e.g., diabodies, triabodies, tetrabodies); minibodies; chelating recombinant antibodies; tribodies or bibodies; intrabodies; nanobodies; small modular immunopharmaceuticals (SMIP), binding-domain immunoglobulin fusion proteins; camelized antibodies; VHH containing antibodies;); and any other polypeptides formed from antibody fragments. The skilled person is aware that the antigen-binding function of an antibody can be performed by fragments of a full-length antibody. Preferably, the binding fragment is a Fab-fragment.

A Fab fragment consists of the VL, VH, CL and CH1 domains. An F(ab′)2 fragment comprises two Fab fragments linked by a disulfide bridge at the hinge region. An Fd is the VH and CH1 domains of a single arm of an antibody. An Fv fragment is the VL and VH domains of a single arm of an antibody.

The beads having immobilized anti-CD3 and anti-CD28 antibodies are commercially available such as Dynabeads (ThermoFisher Scientific #11132D). Also, streptamers® such as (Iba, #6-8901-000) could be used. Preferably beads, i.e. Dynabeads (ThermoFisher Scientific #11132D) are used.

Streptamers® contain Fab-fragments which are bound to Strep-Tactin® multimers. Strep-Tactin® multimers are multimerized Strep-Tactin®, which is a mutein of streptavidin.

Thus, the present method is advantageous since no cytokines and no feeder cells are necessary for stimulation resulting in a cheaper and more efficient stimulation.

Another aspect of the invention refers to a method for TCR independent activation of T cells comprising the steps:
Expressing the CD3-fusion protein as described herein in the T cell,
Stimulating the T cell with a CD3 and a CD28 stimulus.

The method may also comprise the step of deletion of the endogenous TCR. Thus, the method may comprise the following steps:
Expressing the CD3-fusion protein as described herein in the T cell,
Deleting the endogenous TCR of the T cell
Stimulating the T cell with a CD3 and a CD28 stimulus

In specific embodiments of the invention, the method comprises the following steps:
Expressing the CD3-fusion protein as described herein in the T cell,
Deleting the endogenous TCR of the T cell
Stimulating the T cell with a CD3 and a CD28 stimulus
Expression of an exogenous T cell effector molecule

Optionally deletion of the CD3-fusion protein.

The steps deleting the endogenous TCR of the T cell and expressing the CD3-fusion proteins in the T cell may occur substantially in parallel. That means that the time period between the two steps, i.e. between deleting the endogenous TCR of the T cell and the expressing the CD3-fusion protein or between the expressing the CD3-fusion protein and deleting the endogenous TCR of the T cell is less than 12 hours, preferably less than 6 hours, more preferably less than 1 hour, even more preferably less than 10 min, even more preferably less than 5 min, most preferably less than 1 min.

An exogenous T cell effector molecule, is an exogenous antigen specific receptor, more specifically an exogenous antigen specific receptor which is capable of activating the at least one effector function of the T cell and may be a TCR, a chimeric antigen receptor (CAR) or an antibody-coupled T-cell receptor. Preferably, the exogenous antigen specific receptor capable of activating the at least one effector function of the T cell is a TCR.

The effector molecule may be stably or transiently introduced into the T cell. Stable introduction typically may be carried out without limitation by stable transfection, viral transduction or by methods allowing the stable introduction at defined target sites of the genome, for example safe harbor loci or sleeping beauty technologies. Transient introduction typically occurs by transient transfection, preferably by transient transfection of ivtRNA. In preferred embodiments, in particular when the genetically engineered T cell is used for therapy the effector molecule, such as the exogenous TCR α and TCR β chain, may be stably introduced into the T cell. If the genetically engineered T cell is used for characterizing a desired TCR, the T cell is typically transiently transfected, preferably transiently transfected with ivtRNA. A further aspect of the invention relates to a T cell in which both the TCR α chain and the TCR β chain are knocked-out and which comprises at least one exogenous molecule allowing the proliferation of the T cell independent of the endogenous TCR proliferation stimulus in vitro.

Chimeric antigen receptors (CARs) are modularly assembled artificial receptors that confer the specificity of a monoclonal antibody to T cells. The extracellular domain comprises an antibody-derived single-chain variable fragment (scFv) consisting of an immunoglobulin light and heavy chain separated by a flexible linker. The scFv is linked to cytosolic signaling domains via a spacer and a transmembrane domain. The CAR may comprise co-stimulatory signaling domains such as, without limitation CD3ζ, CD27, CD28, CD137, DAP10, FcR, and/or OX40.

The goal of the TCR-independent T cell activation is to keep the T cells devoid of an expressed TCR viable during cell culture, i.e. to expand the T cells in cell culture. Upon the expression of a transgenic/recombinant TCR or chimeric antigen receptor, the T cells elicit cellular effector function (i.e. IFN-γ release) and killing capacity. Typically, the TCR complex is knocked out or its expression is suppressed at one stage of the method of the invention. Therefore, in some embodiments, the T cells comprising the CD3-fusion protein as described herein do not express a functional TCR, or in other word are TCR receptor negative.

Such a TCR-negative T cell comprising the CD3-fusion protein as described herein is ready to use for the introduction of exogenous receptors which can activate the immune effector functions of the recipient cell.

In some embodiments, the cell is isolated or non-naturally occurring.

In specific embodiments, the cell may comprise the nucleic acid encoding the CD3-fusion protein as described herein or the vector comprising said nucleic acid.

In the cell the above described vector comprising a nucleic acid sequence coding for the above described CD3-fusion protein may be introduced or ivtRNA coding for said CD3-fusion protein may be introduced. The cell may be a peripheral blood lymphocyte such as a T cell. The transduction of primary human T cells with a lentiviral vector is, for example, described in Cribbs “simplified production and concentration of lentiviral vectors to achieve high transduction in primary human T cells” BMC Biotechnol. 2013; 13: 98.

The term “transfection” and “transduction” are interchangeable and refer to the process by which an exogenous nucleic acid sequence is introduced in a host cell, e.g. in a eukaryotic host cell. It is noted that introduction or transfer of nucleic acid sequences is not limited to the mentioned methods but can be achieved by any number of means including electroporation, microinjection, gene gun delivery, lipofection, superfection and the mentioned infection by retroviruses or other suitable viruses for transduction or transfection.

In some embodiments, the cell is a peripheral blood lymphocyte (PBL) or a peripheral blood mononuclear cell (PBMC). The cell may be a natural killer cell or a T cell. Preferably, the cell is a T cell. The T cell may be a CD4+ or a CD8+ T cell. In some embodiments the cell is a stem cell like memory T cell.

Stem cell-like memory T cells (TSCM) are a less-differentiated subpopulation of CD8+ T cells, which are characterized by the capacity of self-renewal and to persist long-term. Once these cells encounter their antigen in vivo, they differentiate further into central memory T cells (TCM), effector memory T cells (TEM) and terminally differentiated effector memory T cells (TEMRA) with some TSCM remaining quiescent (Flynn et al., Clinical & Translational Immunology (2014). These remaining TSCM cells show the capacity to build a durable immunological memory in vivo and therefore are considered an important T cell subpopulation for adoptive T cell therapy (Lugli et al., Nature Protocols 8, 33-42 (2013) Gattinoni et al., Nat. Med. 2011 October; 17(10): 1290-1297). Immune-magnetic selection can be used in order to restrict the T cell pool to the stem cell memory T cell subtype see (Riddell et al. 2014, Cancer Journal 20(2): 141-44)

The invention also refers to the T cell may for us as a medicament. In particular, the invention refers to a T cell for the treatment of cancer or viral diseases.

Deletion of TCR

It is clear to the skilled person, that the generation of an efficient knock-out strategy for heterodimer cell surface proteins, such as a TCR, is crucial for the generation of a recipient cell.

The double knock-out of both chains of the TCR may be achieved by the following steps:
(a) knock-out of the endogenous TCR α chain,
(b) selection for cells devoid of a functional TCR,
(c) knock-out of the endogenous TCR β chain,
(d) transient expression of a TCR α chain,
(e) selection for cells devoid of a functional TCR;

or is alternatively achieved by the following steps:
(a) knock-out of the endogenous TCR β chain,
(b) selection for cells devoid of a functional TCR,
(c) knock-out of the endogenous TCR α chain,
(d) transient expression of a TCR β chain,
(e) selection for cells devoid of a functional TCR.

In a particular embodiment, the double knock-out of both TCR α chain and TCR β chain may be achieved by the following steps:
(a) knock-out of the endogenous TCR β chain,
(b) selection for cells devoid of a functional TCR,
(c) knock-out of the endogenous TCR α chain,
(d) transient expression of a TCR β chain,
(e) selection for cells devoid of a functional TCR.

The skilled person understands that this strategy of consecutive knock-out of TCR α chain and TCR β chain is a preferred embodiment and thus not limiting the disclosure of the present application. Alternatively, the double knock-out could also be achieved by a simultaneous knock-out of TCR α chain and TCR β chain.

In step (d) either the pre-TCR α chain or a mature α chain may be transiently expressed. The pre-TCR α chain is a surrogate TCR α chain (also called invariant pTα chain), which is expressed during the development of a T cell. The terms “mature form of the TCR α chain” or “mature TCR α chain” refer to TCR α chain sequences that usually occur after TCR α chain gene arrangement and excludes the pre-TCR α-chain.

Preferably in step (d) “transient expression of a TCR α chain” a mature form of the TCR α chain is transiently expressed.

The term “recipient cell” as used herein refers to a T cell that does not express a functional TCR due to double knock-out of both endogenous TCR α and TCR β chain, and which is able to proliferate independent of an endogenous TCR proliferation stimulus.

Knock-out and double knock-out of (endogenous) TCR α and β chains can be achieved for example by molecular cloning tools comprising Zn finger nucleases (ZFNs), Transcription Activator-like Effector Nucleases (TALENs) and by the clustered regulatory interspaced short palindromic repeat (CRISPR) system, radiation or chemical mutagenesis. The term double knock-out means that the productively rearranged alleles coding for both the TCR α chain and the TCR β chain are made non operative, so that neither a TCR α chain nor a TCR β chain is present in the double knock-out cell. That means that the double knock-out cell not only lacks the functional expression of both TCR α chain and TCR β chain on the cell surface, but it also means that neither the functional endogenous TCR α chain nor the functional endogenous TCR β chain are present in the cell (e.g. ER).

The terms “functional endogenous TCR α chain”, “functional TCR α chain” and “functional TCR chain”, as used herein, mean that the functional TCR α chain is able to pair with a functional TCR β chain and therefore may be expressed at the cell surface. Accordingly, a TCR α chain which is not functional refers to a TCR α chain which is not able to pair with a functional TCR β chain and therefore the TCR α chain will not be expressed at the cell surface.

The terms “functional endogenous TCR β chain”, “functional TCR β chain” and “functional TCR chain”, as used herein, mean that the functional TCR β chain is able to pair with a functional TCR α chain and therefore may be expressed at the cell surface. Accordingly, a TCR β chain which is not functional refers to a TCR β chain which is not able to pair with a functional TCR α chain and therefore the TCR β chain will not be expressed at the cell surface.

ZFNs and TALENs are composed of an adjustable, sequence-specific DNA binding domain and a non-specific DNA cleavage domain. By introducing directed DNA double-strand breaks and stimulating error-prone non-homologous end joining or homology-directed repair mechanisms, they provide excellent tools for genetic manipulation. The DNA binding domain of TALENs is characterized by a central repeat domain of variable length, while each repeat typically consists of 34 amino acids. The specificity of the DNA binding domain depends on an adjacent pair of hypervariable amino acids at the positions 12 and 13 in each repeat, which have been termed repeat-variable di-residues (RVDs). The sequence specificity is governed by a simple code, where each RVD independently specifies one base pair in the DNA. The four most common RVDs preferentially associate with one of the four bases in the DNA (NG=T, HD=C, NI=A, NN=G).

The skilled person is aware of different protocols for the construction of TALENs. Most strategies are based on Golden Gate cloning that allows the simultaneous assembly of multiple DNA fragments in an ordered fashion (Cermak et al., 2011, Efficient design and assembly of custom TALEN and other TAL effector-based constructs for DNA targeting. Nucleic acids research, 39(12), e82; Li et al., 2011, Modularly assembled designer TAL effector nucleases for targeted gene knockout and gene replacement in eukaryotes. Nucleic acids research, 39(14), 6315-25; Morbitzer et al., 2011, Assembly of custom TALE-type DNA binding domains by modular cloning. Nucleic acids research, 39(13), 5790-9, 2011; Sanjana, et al. 2012; A transcription activator-like effector toolbox for genome engineering. Nature protocols, 7(1), 171-92.). The method is based on the use of type IIS restriction enzymes that enable one to perform digestion and ligation in a single reaction mixture. Type IIS restriction enzymes are characterized by their property of cleaving outside the recognition site to create unique 4 bp overhangs. When used for cloning of plasmids, the correct ligation is secured by the unique overhangs and the elimination of the recognition site upon correct assembly of fragments (Engler et al., 2009 Golden gate shuffling: a one-pot DNA shuffling method based on type IIs restriction enzymes. PloS one, 4(5), e5553). In this application, the method of Cermak et al. (2011) was used for the construction of TALENs. Commercial kits, for example EZ-TAL™ (System Bioscience) or FastTALEN™ (Sidansai Biotechnology Co., LTD) as well as commercial services for example offered by Thermo Fisher Scientific or GeneCopoeia are available for TALEN assembly.

In specific embodiments, the double knock-out is obtained by TALENs. The TALENs target region of the TCR α chain may be in the variable AV segment or in the constant AC segment. The TALENs target region of the TCR β chain may be in the variable BV segment or in the constant BC segment. In certain embodiments, the TALENs target region of the TCR α chain is in the constant AC segment and/or wherein the TALENs target region of the TCR β chain is in the constant BC segment. In specific embodiments, the TALENs target region of the TCR α chain is in the constant AC segment and the TALENs target region of the TCR β chain is in the constant BC segment.

An alternative to ZFNs and TALENs is provided by the CRISPR system. In bacteria, the CRISPR system provides acquired immunity against invading foreign DNA via RNA-guided DNA cleavage when using the CRISPR system, two components have to be introduced into cells to perform genome editing: a nuclease (commonly Cas9) and a guided RNA (gRNA). The gRNA consists of CRISPR RNA (crRNA) part and a transactivating CRISPR RNA (tracrRNA) part. Twenty nucleotides at the 5′ end of the gRNA direct the nuclease e.g. Cas9 to a specific target DNA site, which must lie immediately 5′ of a protospacer adjacent motif (PAM) sequence. The CRISPR/Cas9 system may be used to alter genes in a variety of species including mice, rats and human cell lines (see for example Sander and Joung, 2014, Improving CRISPR-Cas nuclease specificity using truncated guide RNAs, Nat Biotechnol. 2014 March; 32(3): 279-284).

The target region of the TCR α chain may be selected in the constant AC segment and/or the target region of the TCR β chain may be selected in the constant BC segment. Thereby any TCR α chain of any specificity may be targeted and/or any TCR β chain of any specificity may be targeted. Thus, it is not necessary to select different target regions for different TCRs having different specificity, for example TCRs having different variable chains. However, if the knock-out is carried out in a specific cell clone of which the type of the variable TCR α chain and the type of the variable TCR β chain is known, the target region of the TCR α chain may be selected in the variable AV segment and/or the target region of the TCR β chain may be selected in the variable BV segment.

Alternatively, the knock-out and/or double knock-out may be achieved by radiation or chemical mutagenesis. Since the knock-out by radiation or chemical mutagenesis is not targeted, the treated cells have to be efficiently selected for mutants that do not express a functional TCR at the cell surface. Therefore, the method of the present invention, i.e. selection of knock-out cells based on presence of the TCR at the cell surface in combination with the transient expression of the complementary TCR chain after the second knock-out is very useful in combination with the random mutation strategies.

In addition, in particular if the recipient cells are used in vivo, the radiated cells may be functionally analyzed in order to select TCR knock-out cells that are not affected by other mutations leading for example to a loss of T cell effector function.

The term “endogenous TCR α and TCR β chain” refers to TCR α and β chains, which are inherent to a T cell.

Although deletion of the TCR α chain leads to the loss of the TCR on the cell surface of mature T cells 7-10 d after deletion, small amounts of surface-bound TCR β chain were expressed over a long period of time (Polic et al., How αβ T cells deal with induced TCRα ablation. Proceedings of the National Academy of Sciences, 98(15), 8744-8749.2001). Additionally, when an exogenous TCR is introduced in single knock-out mutants, the presence of an endogenous TCR chain can lead to the expression of mixed TCR heterodimers with unknown specificity (Sommermeyer et al., 2006, Designer T cells by T cell receptor replacement; European Journal of Immunology, 36(11), 3052-9.). Thus, knocking out both TCR chains has the advantage of preventing even low-level expression of endogenous TCR chains on the cell surface and the potential formation of mixed TCR heterodimers upon expression of exogenous TCR chains.

The transient expression may be for example carried out by expression of ivtRNA.

In preferred embodiments, the TCR α chain transiently expressed in step (d) is the mature form of the TCR α chain.

The skilled person understands from this example of a knock-out of a protein complex comprising three different proteins that by application of this principle also a knock-out of surface protein complexes comprising more than 3 proteins can be generated.

The term “devoid of a TCR at the cell surface” refers to cells that do not express the TCR α/TCR β heterodimer on the cell surface. Whether a T cell expresses a TCR at the cell surface can be detected for example based on antibodies binding to proteins of the TCR complex and flow cytometry.

One embodiment of the application refers to the provision of a method for obtaining a TCR recipient cell. In the resulting TCR recipient cell both, the endogenous TCR α and TCR β chains are knocked out.

The term “transient expression of a TCR α chain”, “transient expression of a mature TCR α chain” or “transient expression of a TCR β chain” refers to the non-stable expression of the TCR α chain or TCR β chain. In certain embodiments the transient expression occurs via the transfection of the cell with ivtRNA. The transient expression of the TCR α chain and TCR β chain after the second knock-out step allows distinguishing between the cells in which one of the two TCR chains is still present and has the advantage that the transiently expressed TCR chain is not permanently expressed in the cell. Hence, when the product of the ivtRNA is no longer expressed and the expressed product is degraded, the cell is devoid of a TCR chain which is transiently expressed.

Therefore, after selection of the cells that are devoid of a TCR at the cell surface in step (e) and when the ivtRNA is no longer expressed and the expressed product is degraded, the cell neither expresses a functional TCR α chain nor a TCR β chain.

Usually, the CD3-fusion protein allows the proliferation of the T cell independent of the endogenous TCR proliferation stimulus in vitro for at least 5, for at least 10, for at least 20, for at least 30, for at least 40, for at least 50, for at least 60, for at least 70, for at least 80, for at least 90, for at least 100, for at least 120, for at least 140, for at least 160, for at least 180, for at least 200, for at least 220, for at least 240, for at least 260, for at least 280, for at least 300 or more generations. This means that a substantial part of the population of cells divide for at least 30, for at least 40, for at least 50, for at least 60, for at least 70, for at least 80, for at least 90, for at least 100, for at least 120, for at least 140, for at least 160, for at least 180, for at least 200, for at least 220, for at least 240, for at least 260, for at least 280, for at least 300 or more times.

In order to selectively kill the therapeutic cells for example if the patient experiences negative side effects, a molecule comprising for example a caspase domain can be introduced into the recipient cell which induces apoptosis upon ligand binding.

Further, in order to avoid an immune response of the patient to the engineered T cell immunogenic surface proteins may be knocked out in the recipient cell. Therefore, the expression of the MHC I and MHC II complex molecules on the cell surface may be prohibited, for example by knock-out. For example, the MHC I complex may be knocked-out in the recipient cell, e.g. by disrupting both alleles of beta-2 microglobulin (B2M).

The T cell may express CD8 and/or CD4 or may be devoid of both CD8 and CD4.

In certain embodiments the T cell expresses CD4. In other embodiments the T cell may express CD8.

“T cell effector function” refers inter alia to the release of cytokines and/or cytotoxic effector proteins, such as perforine, granzyme and granulysin, and/or the expression of the Fas-ligand. The T cell effector functions are described in detail in Janeway, Immunobiology: The Immune System in Health and disease, 2011. Different T cell types exhibit different T cell effector functions.

For example, IFN-γ is released by both CD4+ of the T_{H}1 type and CD8+ T cells. Therefore, the release of IFN-γ is a common test for T cell effector function. The skilled person is aware of different techniques for the measurement of IFN-γ, such as IFN-γ enzyme-linked immunosorbent assay (ELISA) (BD OptEIA™)

The term “exogenous” refers to molecules that have been transferred by any of the genetic engineering techniques into a respective cell.

The present application also contemplates the use of a genetically engineered T cell for testing and characterization of exogenous effector molecules, in particular exogeneous TCRs. The application contemplates the investigation of TCR peptide sensitivities, multimer binding characteristics and functionalities in cytotoxicity and cytokine release assays. A TCR of interest that has to be characterized may be introduced into the recipient cell. The thereby generated genetically engineered T cell carrying the TCR of interest can be used for testing and characterization of the exogenous TCR.

In a specific embodiment, the T cell of the invention has a normal karyotype. In particular when the recipient cell is used for therapy it is preferred that the recipient cell has a normal karyotype and is not altered to have an abnormal karyotype, since cells having an abnormal karyotype are very likely to progress to cancerous cells. The term “normal karyotype” refers to a state of those cells lacking any visible karyotype abnormality detectable for example with chromosome banding analysis or Fluorescence In Situ Hybridization (FISH).

### EXAMPLES

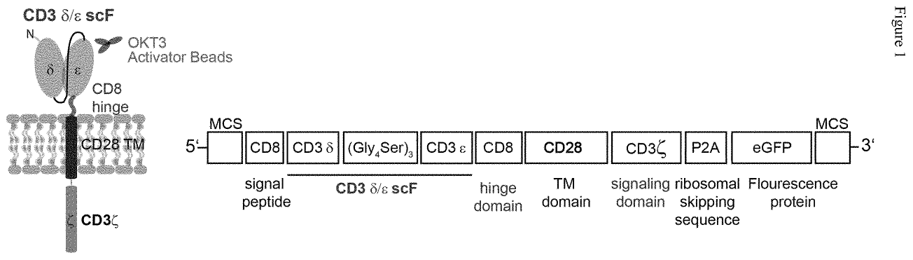

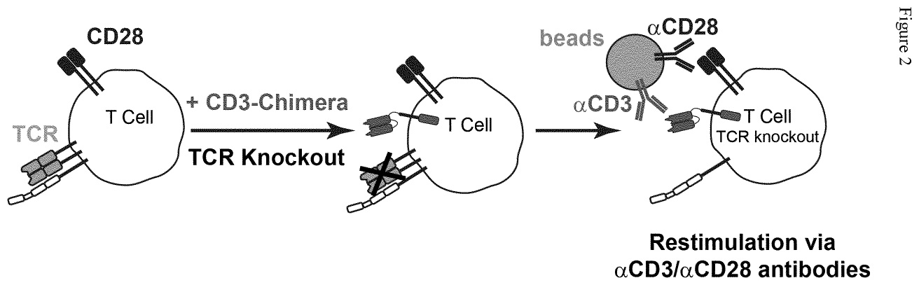

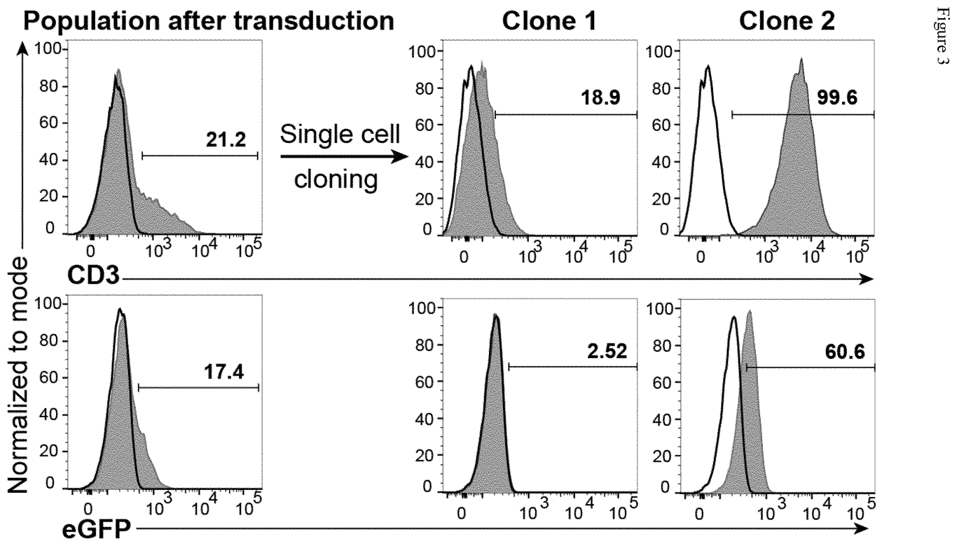

Correct expression and folding of the construct were verified in TCR-deficient Jurkat-76 cells by detection of the CD3-fusion protein as described in FIG. 1 via α-CD3 antibodies and eGFP expression (FIG. 3). Transduced Jurkat-76 cells were analyzed for the expression of CD3-fusion protein and eGFP expression 7 days after transduction by staining with anti-CD3 antibody and subsequent flow cytometric analysis. Generated single cell clones were again evaluated for CD3-fusion protein and eGFP expression by anti-CD3 antibody staining followed by flow cytometric analysis 2 weeks after FACS. The generated single cell clones demonstrated that expression levels of CD3-Chimera could vary in different clones.

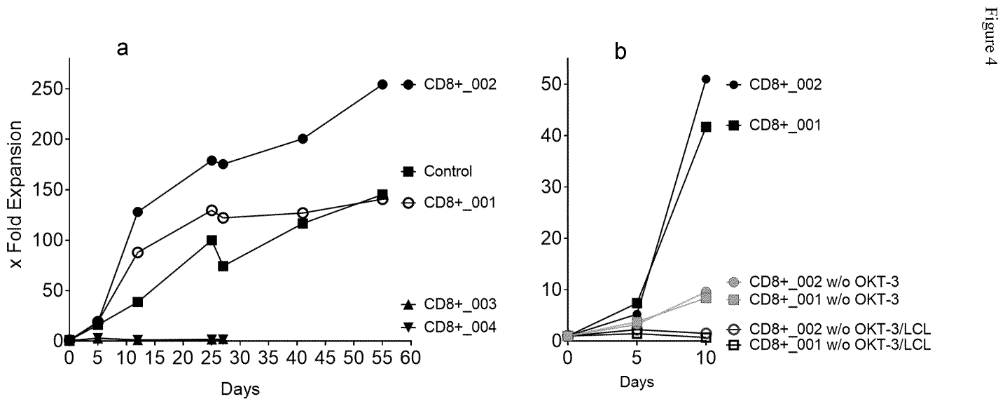

TCR-negative T cells from two donors were generated and simultaneously transduced with the CD3-fusion protein to allow the expansion of the cells via α-CD3 antibody stimulation. The isolated clones, CD8+_001 and CD8+_002, were expanded in vitro and showed a very high proliferation-rate independent of the endogenous TCR (FIG. 4a). The high expansion rate of these T cell clones could only be achieved in the presence of anti-CD3 antibody (OKT-3), which demonstrated that binding of α-CD3 antibody to the CD3-Chimera construct resulted in the activation of these TCR-negative T cells (FIG. 4b).

The universal recipient cells that were generated using the CD3-fusion protein allow the testing of transgenic TCR (T58, D115, as disclosed in WO2010058023A1 and Wilde, S., D. Sommermeyer, B. Frankenberger, M. Schiemann, S. Milosevic, S. Spranger, H. Pohla, W. Uckert, D. H. Busch, and D. J. Schendel. 2009.

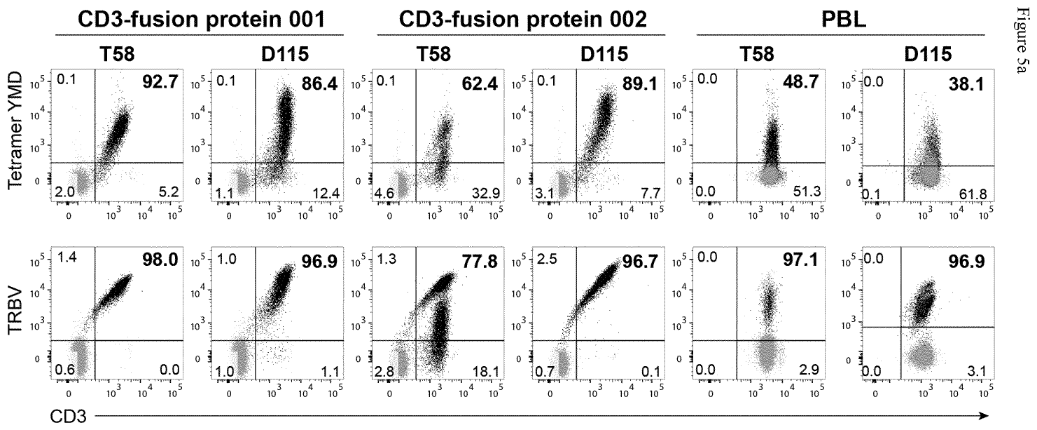

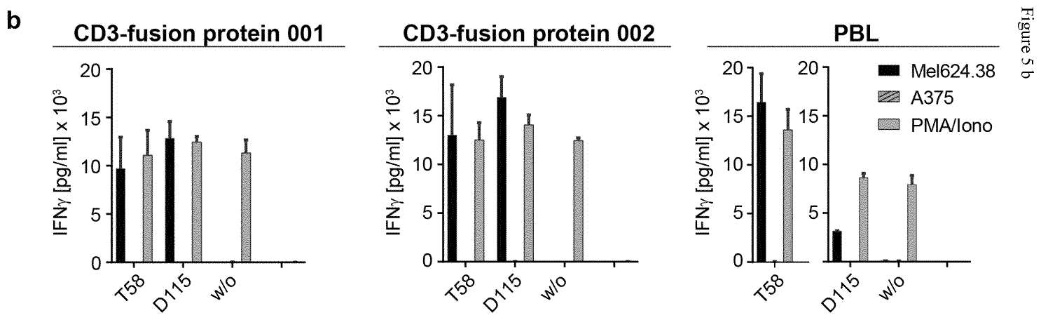

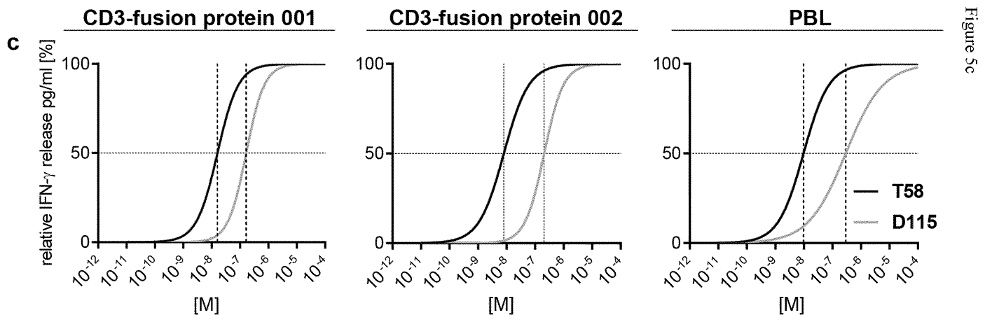

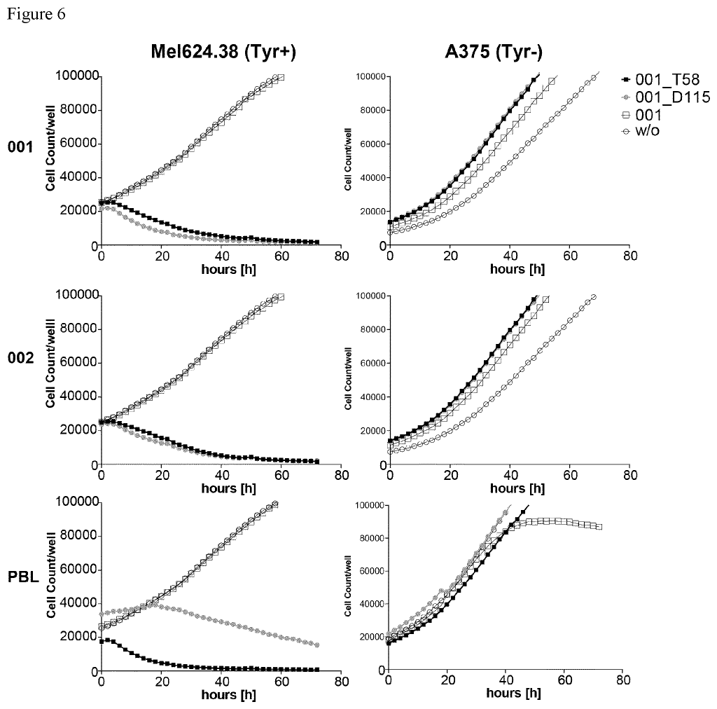

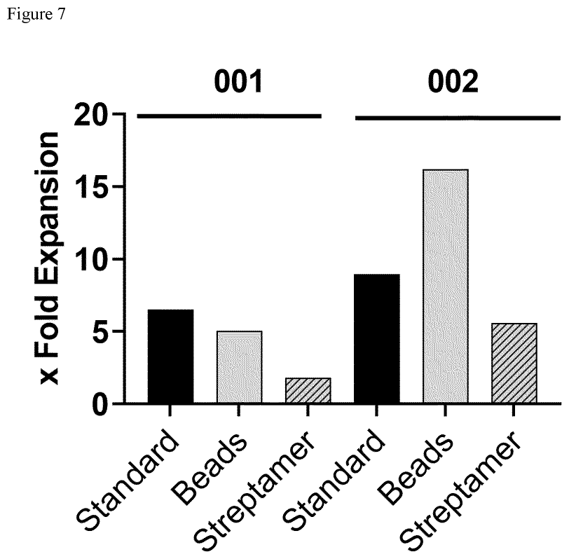

Dendritic cells pulsed with RNA encoding allogeneic MHC and antigen induce T cells with superior antitumor activity and higher TCR functional avidity. Blood 114: 2131-2139) expression, specificity, functional avidity (FIGS. 5a-5c) and killing capacity (FIG. 6) without any background activation of the cells. Isolated T cell clones (CD8+_001 and CD8+_002) and PBL transduced either with T58 or D115, respectively, were stained with α-CD3 and α-TCR-Vβ antibodies specific (TRBV antibody, TCR-Vß23, T58, AF23, IgG1 mouse PE; TCR-Vß8, D115, 56C5.2, IgG2a mouse, both Beckman Coulter) for the respective transgenic TCR β chain and tetramer comprising the YMDGTMSQV (YMD, source: immunAware, Copenhagen, Denmark) peptide (FIG. 5a). After enrichment by FACS, cells were subsequently analyzed by flow cytometric analysis. Untransduced CD8+_001 and CD8+_002 T cell clones and untreated PBL were used as respective negative controls. IFN-γ was measured by ELISA of supernatants derived from the same co-cultures used in the killing assay 20 hours after incubation of transduced T cells (CD8+_001, CD8+_002 or PBL) with target cells at an E:T ratio of 2:1. Positive control comprised effector cells activated with 750 ng/mL PMA and 5 ng/mL ionomycin (PMA/Iono). IFN-γ release of transduced PBL was determined in two independent experiments. Untransduced cells (w/o) served as negative control. Target cells comprised tyrosinase-positive (Mel624.38) and tyrosinase-negative tumor cells (A273) (FIG. 5b). K562_A2_CD86 cells were loaded with decreasing amounts of peptide (10⁻⁴ to 10⁻¹² M) and co-cultured with T58 or D115-expressing T cells, respectively, at a fixed E:T ratio of 2:1. IFN-γ release was determined 20 hours later by IFN-y ELISA and relative IFN-γ release was calculated by setting maximal IFN-γ release to the reference value of 100%. Lower values were calculated according to this reference. Functional avidity of TCR-transduced T cell clones (CD8+_001, CD8+_002) and PBL is plotted in FIG. 5c) as relative IFN-γ release in response to decreasing peptide concentrations. Values were derived from biological duplicates.

In a comparative experiment, isolated clones, CD8⁺_001 and CD8⁺_002, were expanded in vitro under activation with either feeder (LCL) cells and OKT-3 antibody (indicated as Standard), CD3/CD28 streptamer® (Iba, #6-8901-000) or with microspheric beads with α-CD3 and α-CD28 antibodies (Thermo Fisher Scientific, #11132D) immobilized thereon. Cells were expanded for 2 to 3 days. Cells were stimulated over a period of 15 days. On day 15, cell number was determined. Killing assay was conducted using the IncuCyte® ZOOM System to monitor killing (cytolysis) of tyrosinase-positive (Me1624.38) and −negative (A375) tumor cells labeled with IncuCyte® NucLight Red dye over 72 hours with E:T ratio of 2:1. Respective tumor cells alone served as control for proliferation in the absence of effector cells. Untransduced effector cells served as control to estimate background killing in the absence of transgenic TCRs. Cell count/well was determined by using the IncuCyte® ZOOM Software 2016B to analyze quadruplicates of each approach. Although, further costimulatory molecules of the feeder cells are missing, stimulation with streptamer® or beads allow significant activation even on a comparable or better level.

Items

Item 1. A CD3-fusion protein comprising:
a CD3ε ectodomain,
a CD3δ ectodomain or CD3γ ectodomain,
a transmembrane domain, and
a CD3ζ domain.

Item 2. CD3-fusion protein according to item 1, wherein the transmembrane domain is a CD28 transmembrane domain.

Item 3. CD3-fusion protein according to items 1 or 2, wherein the CD3 heterodimer is linked to the transmembrane domain by a hinge domain.

Item 4. CD3-fusion protein according to items 1 to 3 wherein the hinge domain linking the CD3ε ectodomain and CD3δ ectodomain or CD3γ ectodomain to the transmembrane domain is selected from the group consisting of IgG hinge domain, CD 28 hinge domain or CD8 hinge domain.

Item 5. CD3-fusion protein according to item 4, wherein the hinge domain is a CD8 hinge domain.

Item 6. CD3-fusion protein according to the preceding items, wherein the fusion protein further comprises a CD8 signal peptide domain.

Item 7. CD3-fusion protein according to the preceding items, wherein the CD3ζ ectodomain and the CD3ε ectodomain or the CD3ε ectodomain and the CD3γ ectodomain are connected by a linker containing at least 5 amino acids.

Item 8. CD3-fusion protein according to item 7, wherein the amino acids are selected from the group of glycine and serine residues.

Item 9. CD3-fusion protein according to item 8, wherein the fusion protein is capable of activating a TCR-negative T cell in which said fusion protein is expressed, when said TCR-negative T cell is contacted with an CD3 activating stimulus and CD28 activating stimulus.

Item 10. CD3-fusion protein according to item 9, wherein the CD3 activating stimulus is an anti-CD3 antibody

Item 11. CD3-fusion protein according to items 9 or 10, wherein the CD 28 activating stimulus is an anti CD28 antibody

Item 12. CD3-fusion protein according to the preceding items, wherein the fusion protein comprises p1 a CD8 signal peptide domain,
a CD3δ ectodomain and a CD3ε ectodomain,
a CD8 hinge domain,
a CD28 transmembrane domain,
a CD3ζ domain.

Item13. CD3-fusion protein according to the preceding items 6 to 12, wherein the CD8 signal peptide comprises an amino acid sequence which is at least 80% identical to SEQ ID NO: 1.

Item 14. CD3-fusion protein according to the preceding items 6 to 12, wherein the CD8 signal peptide comprises an amino acid sequence which is SEQ ID NO: 1.

Item 15. CD3-fusion protein according to the preceding items, wherein the CD3δ ectodomain comprises an amino acid sequence which is at least 80% identical to SEQ ID NO: 2.

Item 16. CD3-fusion protein according to the preceding items, wherein the CD3δ ectodomain comprises an amino acid sequence which is SEQ ID NO: 2.

Item 17. CD3-fusion protein according to the preceding items, wherein the CD3γ ectodomain comprises an amino acid sequence which is at least 80% identical to SEQ ID NO: 3.

Item 18. CD3-fusion protein according to the preceding items, wherein the CD3γ ectodomain comprises an amino acid sequence which is SEQ ID NO: 3.

Item 19. CD3-fusion protein according to the preceding items, wherein the CD3ε ectodomain comprises an amino acid sequence which is at least 80% identical to SEQ ID NO: 4.

Item 20. CD3-fusion protein according to items 3 to 14, wherein the CD3ε ectodomain comprises an amino acid sequence which is SEQ ID NO: 4.

Item 21. CD3-fusion protein according to the preceding items, wherein the linker connecting the CD3δ domain and the CD3ε domain or the CD3δ domain and the CD3γ domain comprises an amino acid sequence which is at least 80% identical to SEQ ID NO: 5.

Item 22. CD3-fusion protein according to the preceding items, wherein the linker connecting the CD3δ ectodomain and the CD3ε ectodomain or the CD3δ ectodomain and the CD3γ ectodomain comprises an amino acid sequence which is SEQ ID NO: 5.

Item 23. CD3-fusion protein according to the preceding items, wherein the CD8 hinge domain comprises an amino acid sequence which is at least 80% identical to SEQ ID NO: 6.

Item 24. CD3-fusion protein according to the preceding items, wherein the CD8 hinge domain, comprises an amino acid sequence which is SEQ ID NO: 6.

Item 25. CD3-fusion protein according to the preceding items, wherein the transmembrane domain comprises an amino acid sequence which is at least 80% identical to SEQ ID NO: 7.

Item 26. CD3-fusion protein according to the preceding items, wherein the transmembrane domain comprises an amino acid sequence which is SEQ ID NO: 7.

Item 27. CD3-fusion protein according to the preceding items, wherein the CD3ζ domain comprises an amino acid sequence which is at least 80% identical to SEQ ID NO: 8.

Item 28. CD3-fusion protein according to the preceding items, wherein the CD3ζ domain, comprises an amino acid sequence which is SEQ ID NO: 8.

Item 29. CD3-fusion protein according to the preceding items, wherein the fusion protein comprises an amino acid sequence which is at least 80% identical to SEQ ID NO: 11.

Item 30. CD3-fusion protein according to the preceding items, wherein the fusion protein comprises an amino acid sequence which is SEQ ID NO: 11.

Item 31. CD3-fusion protein according to the preceding item, wherein the order of the domains in the direction of N- to C-terminus of the fusion protein is CD3ζ ectodomain or CD3γ ectodomain, linker, CD3ε ectodomain, hinge domain, CD28 transmembrane domain, CD3ζ domain.

Item 32. CD3-fusion protein according to the preceding items, wherein the CD3 fusion protein further comprises a co-stimulatory molecule selected from the group consisting of CD28, OX40, ICOS and CD28.

Item 33. Nucleic acid molecule containing a sequence which encodes for the fusion protein described in items 1 to 32.

Item 34. The nucleic acid molecule of item 33, wherein the vector further comprises a sequence encoding a fluorescence protein.

Item 35. The nucleic acid molecule of item 34, wherein between the sequence encoding the CD3-fusion protein of claims 1 to 32 and the sequence encoding the fluorescence protein is a ribosomal skipping sequence.

Item 36. Use of the fusion protein according to item 1 to 32 or of the nucleic acid molecule according to items 33 to 35 for activation of TCR-negative T cells by an CD3 stimulus and a CD28 stimulus.

Item 37. Use according to item 36, wherein the CD3 stimulus is an activating anti-CD3 antibody or a binding fragment thereof.

Item 38. Use according to item 36 to 37, wherein the CD28 stimulus is an activating anti-CD28 antibody or a binding fragment thereof.

Item 39. Method for TCR independent activation of T cells comprising the steps:
Expressing the fusion protein of items 1 to 32,
Stimulating the T cells with a CD3 and a CD28 stimulus.

Item 40. Method of item 39 comprising the steps:
Expressing the fusion protein of items 1 to 32,
Deleting the TCR
Stimulating the T cells with a CD3 and a CD28 stimulus.

Item 41. Use according to item 38 and method according to item 39 or 40, wherein the CD3 and the CD28 stimulus is a composition comprising an anti-CD3 and an anti-CD28 antibody or binding fragments thereof.

Item 42. Use according to item 38 and method according to item 41, wherein the composition comprising anti-CD3 and anti-CD28 antibodies or binding fragments thereof is immobilized.

Item 43. Use according to item 38 and method according to item 42, wherein the composition comprising anti-CD3 and anti-CD28 or binding fragments thereof is immobilized on beads or on a tissue culture vessel surface.

Item 44. Use according to item 38 and method according to item 43, wherein the composition comprising anti-CD3 and anti-CD28 or binding fragments thereof is immobilized on beads.

Item 45. Use according to item 38 and method according to item 39 to 44, wherein no cytokines are necessary for activation of the TCR-negative T cell.

Item 46. Use according to embodiment 38 and method according to any one of embodiments 39 to 45, wherein the T cell is CD28-positive.

Item 47. Use according to item 38 and method according to any one of items 39 to 46, wherein the TCR complex is knocked out or its expression is suppressed.

Item 48. T cell comprising the fusion protein of item 1 to 32.

Item 49. T cell according to item 48, wherein the T cell devoid of the endogenous TCR.

Item 50. T cell according to any one of items 48 and 49 for use as a medicament.

Item 51. T cell according to any one of items 48 and 49 for use in the treatment of cancer or viral diseases.

Item 52. Use of the T cell according to claims 48 and 49 for testing and characterization of exogenous effector molecules.

Item 53. Use according to item 52, wherein effector molecule is a TCR.

1. A CD3-fusion protein comprising:
a CD3ε ectodomain,
a CD3δ ectodomain or CD3γ ectodomain,
a transmembrane domain, and
a CD3ζ domain.

2. CD3-fusion protein according to the preceding claims, wherein the fusion protein comprises
a CD8 signal peptide domain,
a CD3 heterodimer comprising a CD3δ ectodomain and a CD3ε ectodomain,
a CD8 hinge domain,
a CD28 transmembrane domain,
a CD3ζ domain.

3. CD3-fusion protein according to the preceding claims, wherein the CD3δ ectodomain comprises an amino acid sequence which is SEQ ID NO: 2, wherein the CD3ε ectodomain comprises an amino acid sequence which is SEQ ID NO: 4 and wherein the CD3ζ domain comprises an amino acid sequence which is SEQ ID NO: 8.

4. Nucleic acid molecule containing a sequence, which encodes for the fusion protein described in claims 1 to 3.

5. Method for TCR independent activation of T cells comprising the steps:
Expressing the fusion protein of claims 1 to 3 in the T cell,
Stimulating the T cells with a CD3 and a CD28 stimulus.

6. Method of claim 6 comprising the steps:
Expressing the fusion protein of claims 1 to 3 in the T cell,
Deleting the endogenous TCR of the T cell
Stimulating the T cells with a CD3 and a CD28 stimulus.

7. Method according to claim 5 or 6 wherein the CD3 stimulus is an activating anti-CD3 antibody or a binding fragment thereof.

8. Method according to claim 5 or 6, wherein the CD28 stimulus is an activating anti-CD28 antibody or a binding fragment thereof.

9. Method according to claim 5 or 6, wherein the CD3 and the CD28 stimulus is a composition comprising an anti-CD3 and an anti-CD28 antibody or binding fragments thereof.

10. Method according to claim 9, wherein the composition comprising an anti-CD3 and an anti-CD28 antibody or binding fragments thereof is immobilized.

11. Method according to claim 10, wherein the composition comprising an anti-CD3 and an anti-CD28 antibody or binding fragments thereof is immobilized on beads.

12. T cell comprising the fusion protein of claims 1 to 3.

13. T cell according to claim 12, wherein the T cell devoid of the endogenous TCR.

14. T cell according to any one of claims 12 and 13 for use as a medicament.

15. T cell according to any one of claims 12 and 13 for use in the treatment of cancer or viral diseases.

16. Use of the T cell according to claims 12 and 13 for testing and characterization of exogenous effector molecules.


SEQUENCE LISTING (25 sequences)

SEQ ID NO: 1 - protein, 21 aa, homo sapiens
  misc_feature: CD8 signal peptide domain aa
MALPVTALLLPLALLLHAARP

SEQ ID NO: 2 - protein, 71 aa, homo sapiens
  misc_feature: CD3 delta aa
FKIPIEELEDRVFVNCNTSITWVEGTVGTLLSDITRLDLGKRILDPRGIYRCNGTDIYKDKESTVQVHYRM

SEQ ID NO: 3 - protein, 81 aa, homo sapiens
  misc_feature: CD3 gamma aa
QSIKGNHLVKVYDYQEDGSVLLTCDAEAKNITWFKDGKMIGFLTEDKKKWNLGSNAKDPRGMYQCKGSQNKSKPLQVYYRM

SEQ ID NO: 4 - protein, 96 aa, homo sapiens
  misc_feature: CD3 epsilon aa
DGNEEMGGITQTPYKVSISGTTVILTCPQYPGSEILWQHNDKNIGGDEDDKNIGSDEDHLSLKEFSELEQSGYYVCYPRGSKPEDANFYLYLRARV

SEQ ID NO: 5 - protein, 15 aa, Artificial Sequence
  (G4S) 3 linker aa
GGGGSGGGGSGGGGS

SEQ ID NO: 6 - protein, 45 aa, homo sapiens
  misc_feature: CD8 hinge aa
TTTPAPRPPTPAPTIASQPLSLRPEACRPAAGGAVHTRGLDFACD

SEQ ID NO: 7 - protein, 30 aa, homo sapiens
  misc_feature: CD28 transmembrane aa
FWVLVVVGGVLACYSLLVTVAFIIFWVRSK

SEQ ID NO: 8 - protein, 113 aa, homo sapiens
  misc_feature: CD 3 zeta aa
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 22 aa, Artificial Sequence
  Spacer P2A
GSGATNFSLLKQAGDVEENPGP

SEQ ID NO: 10 - protein, 239 aa, Artificial Sequence
  eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 11 - protein, 652 aa, Artificial Sequence
  CD3-Chimera complete sequence aa
MALPVTALLLPLALLLHAARPFKIPIEELEDRVFVNCNTSITWVEGTVGTLLSDITRLDLGKRILDPRGIYRCNGTDIYKDKESTVQVHYRMGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVTTTPAPRPPTPAPTIASQPLSLRPEACRPAAGGAVHTRGLDFACDFWVLVVVGGVLACYSLLVTVAFIIFWVRSKRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 12 - protein, 63 aa, Artificial Sequence
  CD8 signal peptide domain nt (codon optimized)
ATGGCCCTGCCTGTGACAGCTCTGCTGCTGCCTCTGGCCCTGCTGCTGCATGCCGCCAGACCC

SEQ ID NO: 13 - DNA, 213 bp, Artificial Sequence
  CD3 delta nt (codon optimized)
ttcaagatccccatcgaggaactggaagatcgggtgttcgtgaactgcaacaccagcatcacctgggtggaaggcaccgtgggcacactgctgagcgacatcaccagactggacctgggcaagagaatcctggaccccagaggcatctacagatgcaacggcaccgacatctacaaggacaaagaaagcaccgtgcaggtgcactaccggatg

SEQ ID NO: 14 - DNA, 243 bp, Artificial Sequence
  CD3 gamma nt (codon optimized)
cagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatg

SEQ ID NO: 15 - DNA, 288 bp, Artificial Sequence
  CD3 epsilon nt (codon optimized)
gatggcaacgaggaaatgggcggcatcacccagaccccctacaaggtgtcgatatctggcaccaccgtgatcctgacctgcccccagtatcccggcagcgagattctgtggcagcacaacgacaagaacatcggcggcgacgaggacgataagaatatcggctccgatgaggaccacctgagcctgaaagagttcagcgagctggaacagagcggctactacgtgtgctaccccaggggatccaagcccgaggacgccaacttctacctgtacctgcgggccagagtg

SEQ ID NO: 16 - DNA, 45 bp, Artificial Sequence
  (G4S) 3 linker nt (codon optimized)
ggcggaggcggatctggcggcggaggatcagggggagggggctct

SEQ ID NO: 17 - DNA, 135 bp, Artificial Sequence
  CD8 hinge nt (codon optimized)
accaccacccctgctcctagaccacctaccccagcccctacaatcgccagccagcctctgtctctgaggcccgaggcttgtagaccagctgctggcggagccgtgcacaccagaggactggatttcgcctgcgac

SEQ ID NO: 18 - DNA, 90 bp, Artificial Sequence
  CD28 transmembrane nt (codon optimized)
ttctgggtgctggtggtcgtgggcggcgtgctagcttgttacagcctgctcgtgaccgtggccttcatcatcttttgggtgcgcagcaag

SEQ ID NO: 19 - DNA, 339 bp, Artificial Sequence
  CD 3 zeta nt (codon optimized)
cgcgtgaagttcagcagaagcgccgacgcccctgcctaccagcagggacagaaccagctgtacaacgagctgaacctgggtcgacgagaggaatacgacgtgctggacaagcggagaggccgggaccctgagatgggaggaaagccccagcggagaaagaacccccaggaaggcctgtataacgaactgcagaaagacaagatggccgaggcctacagcgagatcggaatgaagggcgagcgcagaagaggcaagggacacgatggactgtaccagggactgagcaccgccaccaaggatacctatgacgccctgcacatgcaggcccttccacctaga

SEQ ID NO: 20 - DNA, 66 bp, Artificial Sequence
  Spacer P2A nt (codon optimized)
ggttccggagccaccaactttagcctgctgaaacaggccggcgacgtggaagagaaccctggacct

SEQ ID NO: 21 - DNA, 720 bp, Artificial Sequence
  eGFP nt (codon optimized)
atggtgtccaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagagcgccatgcctgagggctacgtgcaagagcggaccatcttctttaaggacgacggcaactacaagaccagagccgaagtgaagtttgagggcgacaccctggtcaaccggatcgagctgaagggcatcgacttcaaagaggacggcaacatcctgggccacaagctcgagtacaactacaactcccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgtctaaggaccccaacgagaagagggaccacatggtgctgctcgagtttgtgacagccgccggaatcaccctcggcatggacgagctgtataagtga

SEQ ID NO: 22 - DNA, 1959 bp, Artificial Sequence
  CD3-Chimera complete sequence nt delta/epsilon (codon optimized)
atggccctgcctgtgacagctctgctgctgcctctggccctgctgctgcatgccgccagacccttcaagatccccatcgaggaactggaagatcgggtgttcgtgaactgcaacaccagcatcacctgggtggaaggcaccgtgggcacactgctgagcgacatcaccagactggacctgggcaagagaatcctggaccccagaggcatctacagatgcaacggcaccgacatctacaaggacaaagaaagcaccgtgcaggtgcactaccggatgggcggaggcggatctggcggcggaggatcagggggagggggctctgatggcaacgaggaaatgggcggcatcacccagaccccctacaaggtgtcgatatctggcaccaccgtgatcctgacctgcccccagtatcccggcagcgagattctgtggcagcacaacgacaagaacatcggcggcgacgaggacgataagaatatcggctccgatgaggaccacctgagcctgaaagagttcagcgagctggaacagagcggctactacgtgtgctaccccaggggatccaagcccgaggacgccaacttctacctgtacctgcgggccagagtgaccaccacccctgctcctagaccacctaccccagcccctacaatcgccagccagcctctgtctctgaggcccgaggcttgtagaccagctgctggcggagccgtgcacaccagaggactggatttcgcctgcgacttctgggtgctggtggtcgtgggcggcgtgctagcttgttacagcctgctcgtgaccgtggccttcatcatcttttgggtgcgcagcaagcgcgtgaagttcagcagaagcgccgacgcccctgcctaccagcagggacagaaccagctgtacaacgagctgaacctgggtcgacgagaggaatacgacgtgctggacaagcggagaggccgggaccctgagatgggaggaaagccccagcggagaaagaacccccaggaaggcctgtataacgaactgcagaaagacaagatggccgaggcctacagcgagatcggaatgaagggcgagcgcagaagaggcaagggacacgatggactgtaccagggactgagcaccgccaccaaggatacctatgacgccctgcacatgcaggcccttccacctagaggttccggagccaccaactttagcctgctgaaacaggccggcgacgtggaagagaaccctggacctatggtgtccaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagagcgccatgcctgagggctacgtgcaagagcggaccatcttctttaaggacgacggcaactacaagaccagagccgaagtgaagtttgagggcgacaccctggtcaaccggatcgagctgaagggcatcgacttcaaagaggacggcaacatcctgggccacaagctcgagtacaactacaactcccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgtctaaggaccccaacgagaagagggaccacatggtgctgctcgagtttgtgacagccgccggaatcaccctcggcatggacgagctgtataagtga

SEQ ID NO: 23 - protein, 662 aa, Artificial Sequence
  CD3-Chimera complete sequence aa gamma/epsilon (codon optimized)
MALPVTALLLPLALLLHAARPQSIKGNHLVKVYDYQEDGSVLLTCDAEAKNITWFKDGKMIGFLTEDKKKWNLGSNAKDPRGMYQCKGSQNKSKPLQVYYRMGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVTTTPAPRPPTPAPTIASQPLSLRPEACRPAAGGAVHTRGLDFACDFWVLVVVGGVLACYSLLVTVAFIIFWVRSKRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 24 - DNA, 1989 bp, Artificial Sequence
  CD3-Chimera complete sequence nt gamma/epsilon (codon optimized)
atggccctgcctgtgacagctctgctgctgcctctggccctgctgctgcatgccgccagaccccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgggcggaggcggatctggcggcggaggatcagggggagggggctctgatggcaacgaggaaatgggcggcatcacccagaccccctacaaggtgtcgatatctggcaccaccgtgatcctgacctgcccccagtatcccggcagcgagattctgtggcagcacaacgacaagaacatcggcggcgacgaggacgataagaatatcggctccgatgaggaccacctgagcctgaaagagttcagcgagctggaacagagcggctactacgtgtgctaccccaggggatccaagcccgaggacgccaacttctacctgtacctgcgggccagagtgaccaccacccctgctcctagaccacctaccccagcccctacaatcgccagccagcctctgtctctgaggcccgaggcttgtagaccagctgctggcggagccgtgcacaccagaggactggatttcgcctgcgacttctgggtgctggtggtcgtgggcggcgtgctagcttgttacagcctgctcgtgaccgtggccttcatcatcttttgggtgcgcagcaagcgcgtgaagttcagcagaagcgccgacgcccctgcctaccagcagggacagaaccagctgtacaacgagctgaacctgggtcgacgagaggaatacgacgtgctggacaagcggagaggccgggaccctgagatgggaggaaagccccagcggagaaagaacccccaggaaggcctgtataacgaactgcagaaagacaagatggccgaggcctacagcgagatcggaatgaagggcgagcgcagaagaggcaagggacacgatggactgtaccagggactgagcaccgccaccaaggatacctatgacgccctgcacatgcaggcccttccacctagaggttccggagccaccaactttagcctgctgaaacaggccggcgacgtggaagagaaccctggacctatggtgtccaaaggcgaggaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgctttagcagataccccgaccatatgaagcagcacgacttcttcaagagcgccatgcctgagggctacgtgcaagagcggaccatcttctttaaggacgacggcaactacaagaccagagccgaagtgaagtttgagggcgacaccctggtcaaccggatcgagctgaagggcatcgacttcaaagaggacggcaacatcctgggccacaagctcgagtacaactacaactcccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgtctaaggaccccaacgagaagagggaccacatggtgctgctcgagtttgtgacagccgccggaatcaccctcggcatggacgagctgtataagtga

SEQ ID NO: 25 - protein, 9 aa, Artificial Sequence
  Control peptide
SLLMWITQC